SEQUENCE LISTING (20 sequences)

SEQ ID NO: 1 - protein, 713 aa, Bacillus agaradherens
MSKKTLKRLLALVVVLFILSGSGILDFSITSANAQQATDRSNSVNYSTDGIYQIVTDRFYDGDESNNPSGELYSEGCKNLRKYCGGDWQGIIDKIDDGYLTNMGVTALWISPPVENIFETIDDESGTTSYHGYWARDYKKTNPFFGSTEDFERLIETAHSHDIKIVIDLAPNHTSPADFDNPNYAENGILYDNGNYVSSYSDNSDLFLYNGGTDFSTYEDEIYRNLFDLASFNHINAELNNYLEDAVKKWLDLGIDGIRIDAVAHMPPGWQKAYMDTIYDHRAVFTFGEWFTGPYGNEDYTKFANNSGMSVLDFRFAQTTRNVIGNNNGTMYDIEKMLTDTENDYDRPQDQVTFLDNHDMSRFTNDGESTRTTDIGLALMLTSRGVPTIYYGTEQYMEGDGDPGSRGMMESFGENTDAYKLIQKLAPLRKSNPAYGYGTTKERWINDDVIIYERNFGDNYALIAINRNLNTSYNIQGLQTEMPSNSYDDVLDGLLDGQSIVVDNNGEVNEFQMSPGEVGVWEFEATNVDKPSIGQVGPIIGEAGRTVTISGEGFGSSPGTVQFGSTSAEIVSWNDTVIIITVPNNEAGYHDITVVTEDEQVSNAYEFEVLTADQVTVRFIIDNAETKMGENIFLVGNVHELGNWDPEQSVGRFFNQVVYQYPTWYYDVNVPANTDLEFKFIKIDQDNNVTWQSGANHTYSSPESGTGIIRVDW

SEQ ID NO: 2 - protein, 713 aa, Bacillus agaradherens
MRKKTLKRLLTLVVGLVILSGLSILDFSITSASAQQATDRSNSVNYSTDVIYQIVTDRFYDGDESNNPSGELYSEDCKNLRKYCGGDWQGIIDKIDDGYLTNMGVTALWISPPVENIFETIDDEFGTTSYHGYWARDYKKTNPFFGSTEDFERLIETAHSHDIKIVIDLAPNHTSPADFDNPDYAENGVLYDDGNYLGSYSDDSDLFLYNGGTDFSNYEDEIYRNLFDLASFNHINSELNNYLEDAVKKWLDLGIDGIRIDAVAHMPPGWKKAYMDTIYDHRAVFTFGEWFTGPSGNEDYTKFANNSGMSVLDFRFAQTTRNVIGNNNGTMYDIEKMLTDTENDYDRPQDQVTFLDNHDMSRFTNGGESTRTTDIGLALMLTSRGVPTIYYGTEQYMKGDGDPGSRGMMASFDENTDAYKLIQKLAPLRKSNPAYGYGTTTERWINDDVLIYERHFGENYALIAINRSLNTSYNIQGLQTEMPSNSYDDVLDGLLDGQSIVVDNKGGVNEFQMSPGEVSVWEFEAENVDKPSIGQVGPIIGEAGRTVTISGEGFGSSQGTVHFGSTSAEILSWNDTIITLTVPNNEAGYHDITVVTEDEQVSNAYEFEVLTADQVTVRFIIDNAETKLGENVFLVGNVHELGNWDPEQSVGRFFNQIVYQYPTWYYDVNVPANTDLEFKFIKIDQDNNVIWQSGANQTYSSPESGTGIIRVDW

SEQ ID NO: 3 - protein, 714 aa, Panibacillus macerans
MKSRYKRLTSLALSLSMALGISLPAWASPDTSVDNKVNFSTDVIYQIVTDRFADGDRTNNPAGDAFSGDRSNLKLYFGGDWQGIIDKINDGYLTGMGVTALWISQPVENITSVIKYSGVNNTSYHGYWARDFKQTNDAFGDFADFQNLIDTAHAHNIKVVIDFAPNHTSPADRDNPGFAENGGMYDNGSLLGAYSNDTAGLFHHNGGTDFSTIEDGIYKNLYDLADINHNNNAMDAYFKSAIDLWLGMGVDGIRFDAVKHMPFGWQKSFVSSIYGGDHPVFTFGEWYLGADQTDGDNIKFANESGMNLLDFEYAQEVREVFRDKTETMKDLYEVLASTESQYDYINNMVTFIDNHDMDRFQVAGSGTRATEQALALTLTSRGVPAIYYGTEQYMTGDGDPNNRAMMTSFNTGTTAYKVIQALAPLRKSNPAIAYGTTTERWVNNDVLIIERKFGSSAALVAINRNSSAAYPISGLLSSLPAGTYSDVLNGLLNGNSITVGSGGAVTNFTLAAGGTAVWQYTAPETSPAIGNVGPTMGQPGNIVTIDGRGFGGTAGTVYFGTTAVTGSGIVSWEDTQIKAVIPKVAAGKTGVSVKTSSGTASNTFKSFNVLTGDQVTVRFLVNQANTNYGTNVYLVGNAAELGSWDPNKAIGPMYNQVIAKYPSWYYDVSVPAGTKLDFKFIKKGGGTVTWEGGGNHTYTTPASGVGTVTVDWQN

SEQ ID NO: 4 - protein, 713 aa, Panibacillus macerans
MKKQVKWLTSVSMSVGIALGAALPVWASPDTSVNNKLNFSTDTVYQIVTDRFVDGNSANNPTGAAFSSDHSNLKLYFGGDWQGITNKINDGYLTGMGITALWISQPVENITAVINYSGVNNTAYHGYWPRDFKKTNAAFGSFTDFSNLIAAAHSHNIKVVMDFAPNHTNPASSTDPSFAENGALYNNGTLLGKYSNDTAGLFHHNGGTDFSTTESGIYKNLYDLADINQNNNTIDSYLKESIQLWLNLGVDGIRFDAVKHMPQGWQKSYVSSIYSSANPVFTFGEWFLGPDEMTQDNINFANQSGMHLLDFAFAQEIREVFRDKSETMTDLNSVISSTGSSYNYINNMVTFIDNHDMDRFQQAGASTRPTEQALAVTLTSRGVPAIYYGTEQYMTGNGDPNNRGMMTGFDTNKTAYKVIKALAPLRKSNPALAYGSTTQRWVNSDVYVYERKFGSNVALVAVNRSSTTAYPISGALTALPNGTYTDVLGGLLNGNSITVNGGTVSNFTLAAGGTAVWQYTTTESSPIIGNVGPTMGKPGNTITIDGRGFGTTKNKVTFGTTAVTGANIVSWEDTEIKVKVPNVAAGNTAVTVTNAAGTTSAAFNNFNVLTADQVTVRFKVNNATTALGQNVYLTGNVAELGNWTAANAIGPMYNQVEASYPTWYFDVSVPANTALQFKFIKVNGSTVTWEGGNNHTFTSPSSGVATVTVDWQN

SEQ ID NO: 5 - protein, 683 aa, Thermoanaerobacterium thermosulfurigenes
ASDTAVSNVVNYSTDVIYQIVTDRFVDGNTSNNPTGDLYDPTHTSLKKYFGGDWQGIINKINDGYLTGMGVTAIWISQPVENIYAVLPDSTFGGSTSYHGYWARDFKRTNPYFGSFTDFQNLINTAHAHNIKVIIDFAPNHTSPASETDPTYAENGRLYDNGTLLGGYTNDTNGYFHHYGGTDFSSYEDGIYRNLFDLADLNQQNSTIDSYLKSAIKVWLDMGIDGIRLDAVKHMPFGWQKNFMDSILSYRPVFTFGEWFLGTNEIDVNNTYFANESGMSLLDFRFSQKVRQVFRDNTDTMYGLDSMIQSTASDYNFINDMVTFIDNHDMDRFYNGGSTRPVEQALAFTLTSRGVPAIYYGTEQYMTGNGDPYNRAMMTSFNTSTTAYNVIKKLAPLRKSNPAIAYGTTQQRWINNDVYIYERKFGNNVALVAINRNLSTSYNITGLYTALPAGTYTDVLGGLLNGNSISVASDGSVTPFTLSAGEVAVWQYVSSSNSPLIGHVGPTMTKAGQTITIDGRGFGTTSGQVLFGSTAGTIVSWDDTEVKVKVPSVTPGKYNISLKTSSGATSNTYNNINILTGNQICVRFVVNNASTVYGENVYLTGNVAELGNWDTSKAIGPMFNQVVYQYPTWYYDVSVPAGTTIQFKFIKKNGNTITWEGGSNHTYTVPSSSTGTVIVNWQQ

SEQ ID NO: 6 - protein, 683 aa, Thermoanaerobacter species.
APDTSVSNVVNYSTDVIYQIVTDRFLDGNPSNNPTGDLYDPTHTSLKKYFGGDWQGIINKINDGYLTGMGITAIWISQPVENIYAVLPDSTFGGSTSYHGYWARDFKKTNPFFGSFTDFQNLIATAHAHNIKVIIDFAPNHTSPASETDPTYGENGRLYDNGVLLGGYTNDTNGYFHHYGGTNFSSYEDGIYRNLFDLADLDQQNSTIDSYLKAAIKLWLDMGIDGIRMDAVKHMAFGWQKNFMDSILSYRPVFTFGEWYLGTNEVDPNNTYFANESGMSLLDFRFAQKVRQVFRDNTDTMYGLDSMIQSTAADYNFINDMVTFIDNHDMDRFYTGGSTRPVEQALAFTLTSRGVPAIYYGTEQYMTGNGDPYNRAMMTSFDTTTTAYNVIKKLAPLRKSNPAIAYGTQKQRWINNDVYIYERQFGNNVALVAINRNLSTSYYITGLYTALPAGTYSDMLGGLLNGSSITVSSNGSVTPFTLAPGEVAVWQYVSTTNPPLIGHVGPTMTKAGQTITIDGRGFGTTAGQVLFGTTPATIVSWEDTEVKVKVPALTPGKYNITLKTASGVTSNSYNNINVLTGNQVCVRFVVNNATTVWGENVYLTGNVAELGNWDTSKAIGPMFNQVVYQYPTWYYDVSVPAGTTIEFKFIKKNGSTVTWEGGYNHVYTTPTSGTATVIVDWQP

SEQ ID NO: 7 - protein, 718 aa, Bacillus circulans
MFQMAKRAFLSTTLTLGLLAGSALPFLPASAVYADPDTAVTNKQSFSTDVIYQVFTDRFLDGNPSNNPTGAAYDATCSNLKLYCGGDWQGLINKINDNYFSDLGVTALWISQPVENIFATINYSGVTNTAYHGYWARDFKKTNPYFGTMADFQNLITTAHAKGIKIVIDFAPNHTSPAMETDTSFAENGRLYDNGTLVGGYTNDTNGYFHHNGGSDFSSLENGIYKNLYDLADFNHNNATIDKYFKDAIKLWLDMGVDGIRVDAVKHMPLGWQKSWMSSIYAHKPVFTFGEWFLGSAASDADNTDFANKSGMSLLDFRFNSAVRNVFRDNTSNMYALDSMINSTATDYNQVNDQVTFIDNHDMDRFKTSAVNNRRLEQALAFTLTSRGVPAIYYGTEQYLTGNGDPDNRAKMPSFSKSTTAFNVISKLAPLRKSNPAIAYGSTQQRWINNDVYVYERKFGKSVAVVAVNRNLSTSASITGLSTSLPTGSYTDVLGGVLNGNNITSTNGSINNFTLAAGATAVWQYTTAETTPTIGHVGPVMGKPGNVVTIDGRGFGSTKGTVYFGTTAVTGAAITSWEDTQIKVTIPSVAAGNYAVKVAASGVNSNAYNNFTILTGDQVTVRFVVNNASTTLGQNLYLTGNVAELGNWSTGSTAIGPAFNQVIHQYPTWYYDVSVPAGKQLEFKFFKKNGSTITWESGSNHTFTTPASGTATVTVNWQ

SEQ ID NO: 8 - protein, 718 aa, Bacillus species. 38-2
MFQMAKRVLLSTTLTFSLLAGSALPFLPASAIYADADTAVTNKQNFSTDVIYQVFTDRFLDGNPSNNPTGAAFDGTCSNLKLYCGGDWQGLVNKINDNYFSDLGVTALWISQPVENIFATINYSGVTNTAYHGYWARDFKKTNPYFGTMTDFQNLVTTAHAKGIKIIIDFAPNHTSPAMETDTSFAENGKLYDNGNLVGGYTNDTNGYFHHNGGSDFSTLENGIYKNLYDLADLNHNNSTIDTYFKDAIKLWLDMGVDGIRVDAVKHMPQGWQKNWMSSIYAHKPVFTFGEWFLGSAAPDADNTDFANESGMSLLDFRFNSAVRNVFRDNTSNMYALDSMLTATAADYNQVNDQVTFIDNHDMDRFKTSAVNNRRLEQALAFTLTSRGVPAIYYGTEQYLTGNGDPDNRGKMPSFSKSTTAFNVISKLAPLRKSNPAIAYGSTQQRWINNDVYIYERKFGKSVAVVAVNRNLTTPTSITNLNTSLPSGTYTDVLGGVLNGNNITSSGGNISSFTLAAGATAVWQYTASETTPTIGHVGPVMGKPGNVVTIDGRGFGSAKGTVYFGTTAVTGSAITSWEDTQIKVTIPPVAGGDYAVKVAANGVNSNAYNDFTILSGDQVSVRFVINNATTALGENIYLTGNVSELGNWTTGAASIGPAFNQVIHAYPTWYYDVSVPAGKQLEFKFFKKNGATITWEGGSNHTFTTPTSGTATVTINWQ

SEQ ID NO: 9 - protein, 713 aa, Bacillus species. 1011
MKRFMKLTAVWTLWLSLTLGLLSPVHAAPDTSVSNKQNFSTDVIYQIFTDRFSDGNPANNPTGAAFDGSCTNLRLYCGGDWQGIINKINDGYLTGMGITAIWISQPVENIYSVINYSGVNNTAYHGYWARDFKKTNPAYGTMQDFKNLIDTAHAHNIKVIIDFAPNHTSPASSDDPSFAENGRLYDNGNLLGGYTNDTQNLFHHYGGTDFSTIENGIYKNLYDLADLNHNNSSVDVYLKDAIKMWLDLGVDGIRVDAVKHMPFGWQKSFMATINNYKPVFTFGEWFLGVNEISPEYHQFANESGMSLLDFRFAQKARQVFRDNTDNMYGLKAMLEGSEVDYAQVNDQVTFIDNHDMERFHTSNGDRRKLEQALAFTLTSRGVPAIYYGSEQYMSGGNDPDNRARLPSFSTTTTAYQVIQKLAPLRKSNPAIAYGSTHERWINNDVIIYERKFGNNVAVVAINRNMNTPASITGLVTSLRRASYNDVLGGILNGNTLTVGAGGAASNFTLAPGGTAVWQYTTDATTPIIGNVGPMMAKPGVTITIDGRGFGSGKGTVYFGTTAVTGADIVAWEDTQIQVKIPAVPGGIYDIRVANAAGAASNIYDNFEVLTGDQVTVRFVINNATTALGQNVFLTGNVSELGNWDPNNAIGPMYNQVVYQYPTWYYDVSVPAGQTIEFKFLKKQGSTVTWEGGANRTFTTPTSGTATVNVNWQP

SEQ ID NO: 10 - protein, 712 aa, Bacillus sp. 38-2
MKRFMKLTAVWTLWLSLTLGLLSPVHAAPDTSVSNKQNFSTDVIYQIFTDRFSDGNPANNPTGAAFDGSCTNLRLYCGGDWQGIINKINDGYLTGMGITAIWISQPVENIYSVINYSGVHNTAYHGYWARDFKKTNPAYGTMQDFKNLIDTAHAHNIKVIIDFAPNHTSPASSDDPSFAENGRLYDNGNLLGGYTNDTQNLFHHYGGTDFSTIENGIYKNLYDLADLNHNNSSVDVYLKDAIKMWLDLGVDGIRVDAVKHMPFGWQKSFMSTINNYKPVFNFGEWFLGVNEISPEYHQFANESGMSLLDFPFAQKARQVFRDNTDNMYGLKAMLEGSEVDYAQVNDQVTFIDNHDMERFHTSNGDRRKLEQALAFTLTSRGVPAIYYGSEQYMSGGNDPDNRARIPSFSTTTTAYQVIQKLAPLRKSNPAIAYGSTQERWINNDVIIYERKFGNNVAVVAINRNMNTPASITGLVTSLPQGSYNDVLGGILNGNTLTVGAGGAASNFTLAPGGTAVWQYTTDATAPINGNVGPMMAKAGVTITIDGRASARQGTVYFGTTAVTGADIVAWEDTQIQVKILRVPGGIYDIRVANAAGAASNIYDNFEVLTGDQVTVRFVINNATTALGQNVFLTGNVSELGNWDPNNAIGPMYNQVVYQYPTWYYDVSVPAGQTIEFKFLKKQGSTVTWEGGANRTFTTPTSGTATVNVNWQP

SEQ ID NO: 11 - protein, 713 aa, Bacillus circulans
MKKFLKSTAALALGLSLTFGLFSPAQAAPDTSVSNKQNFSTDVIYQIFTDRFSDGNPANNPTGAAFDGTCTNLRLYCGGDWQGIINKINDGYLTGMGVTAIWISQPVENIYSIINYSGVNNTAYHGYWARDFKKTNPAYGTIADFQNLIAAAHAKNIKVIIDFAPNHTSPASSDQPSFAENGRLYDNGTLLGGYTNDTQNLFHHNGGTDFSTTENGIYKNLYDLADLNHNNSTVDVYLKDAIKMWLDLGIDGIRMDAVKHMPFGWQKSFMAAVNNYKPVFTFGEWFLGVNEVSPENHKFANESGMSLLDFRFAQKVRQVFRDNTDNMYGLKAMLEGSAADYAQVDDQVTFIDNHDMERFHASNANRRKLEQALAFTLTSRGVPAIYYGTEQYMSGGTDPDNRARIPSFSTSTTAYQVIQKLAPLRKCNPAIAYGSTQERWINNDVLIYERKFGSNVAVVAVNRNLNAPASISGLVTSLPQGSYNDVLGGLLNGNTLSVGSGGAASNFTLAAGGTAVWQYTAATATPTIGHVGPMMAKPGVTITIDGRGFGSSKGTVYFGTTAVSGADITSWEDTQIKVKIPAVAGGNYNIKVANAAGTASNVYDNFEVLSGDQVSVRFVVNNATTALGQNVYLTGSVSELGNWDPAKAIGPMYNQVVYQYPNWYYDVSVPAGKTIEFKFLKKQGSTVTWEGGSNHTFTAPSSGTATINVNWQP

SEQ ID NO: 12 - protein, 686 aa, Bacillus species.
APDTSVSNKQNFSTDVIYQIFTDRFSDGNPANNPTGAAFDGSCTNLRLYCGGDWQGIINKINDGYLTGMGITAIWISQPVENIYSVINYSGVNNTAYHGYWARDFKKTNPAYGTMQDFKNLIDTAHAHNIKVIIDFAPNHTSPASSDDPSFAENGRLYDNGNLLGGYTNDTQNLFHHYGGTDFSTIENGIYKNLYDLADLNHNNSSVDVYLKDAIKMWLDLGVDGIRVDAVKHMPFGWQKSFMSTINNYKPVFTFGEWFLGVNEISPEYHQFANESGMSLLDFRFAQKARQVFRDNTDNMYGLKAMLEGSEVDYAQVNDQVTFIDNHDMERFHTSNGDRRKLEQALAFTLTSRGVPAIYYGSEQYMSGGNDPDNRARIPSFSTTTTAYQVIQKLAPLRKSNPAIAYGSTQERWINNDVIIYERKFGNNVAVVAINRNMNTPASITGLVTSLPQGSYNDVLGGILNGNTLTVGAGGAASNFTLAPGGTAVWQYTTDATAPIIGNVGPMMAKPGVTITIDGRGFGSGKGTVYFGTTAVTGADIVAWEDTQIQVKIPAVPGGIYDIRVANAAGAASNIYDNFEVLTGDQVTVRFVINNATTALGQNVFLTGNVSELGNWDPNNAIGPMYNQVVYQYPTWYYDVSVPAGQTIEFKFLKKQGSTVTWEGGANRTFTTPTSGTATMNVNWQP

SEQ ID NO: 13 - protein, 704 aa, Bacillus ohbensis
MKNLTVLLKTIPLALLLFILLSLPTAAQADVTNKVNYTRDVIYQIVTDRFSDGDPSNNPTGAIYSQDCSDLHKYCGGDWQGIIDKINDGYLTDLGITAIWISQPVENVYALHPSGYTSYHGYWARDYKRTNPFYGDFSDFDRLMDTAHSNGIKVIMDFTPNHSSPALETDPSYAENGAVYNDGVLIGNYSNDPNNLFHHNGGTDFSSYEDSIYRNLYDLADYDLNNTVMDQYLKESIKLWLDKGIDGIRVDAVKHMSEGWQTSLMSDIYAHEPVFTFGEWFLGSGEVDPQNHHFANESGMSLLDFQFGQTIRDVLMDGSSNWYDFNEMIASTEEDYDEVIDQVTFIDNHDMSRFSFEQSSNRHTDIALAVLLTSRGVPTIYYGTEQYLTGGNDPENRKPMSDFDRTTNSYQIISTLASLRQNNPALGYGNTSERWINSDVYIYERSFGDSVVLTAVNSGDTSYTINNLNTSLPQGQYTDELQQLLDGNEITVNSNGAVDSFQLSANGVSVWQITEEHASPLIGHVGPMMGKHGNTVTITGEGFGDNEGSVLFDSDFSDVLSWSDTKIEVSVPDVTAGHYDISVVNAGDSQSPTYDKFEVLTGDQVSIRFAVNNATTSLGTNLYMVGNVNELGNWDPDQAIGPMFNQVMYQYPTWYYDISVPAEENLEYKFIKKDSSGNVVWESGNNHTYTTPATGTDTVLVDWQ

SEQ ID NO: 14 - protein, 703 aa, Bacillus species. 1-1
MNDLNDFLKTILLSFIFFLLLSLPTVAEADVTNKVNYSKDVIYQIVTDRFSDGNPGNNPSGAIFSQNCIDLHKYCGGDWQGIIDKINDGYLTDLGITALWISQPVENVYALHPSGYTSYHGYWARDYKKTNPYYGNFDDFDRLMSTAHSNGIKVIMDFTPNHSSPALETNPNYVENGAIYDNGALLGNYSNDQQNLFHHNGGTDFSSYEDSIYRNLYDLADYDLNNTVMDQYLKESIKFWLDKGIDGIRVDAVKHMSEGWQTSLMSEIYSHKPVFTFGEWFLGSGEVDPQNHHFANESGMSLLDFQFGQTIRNVLKDRTSNWYDFNEMITSTEKEYNEVIDQVTFIDNHDMSRFSVGSSSNRQTDMALAVLLTSRGVPTIYYGTEQYVTGGNDPENRKPLKTFDRSTNSYQIISKLASLRQTNSALGYGTTTERWLNEDIYIYERTFGNSIVLTAVNSSNSNQTITNLNTSLPQGNYTDELQQRLDGNTITVNANGAVNSFQLRANSVAVWQVSNPSTSPLIGQVGPMMGKAGNTITVSGEGFGDERGSVLFDSTSSEIISWSNTKISVKVPNVAGGYYDLSVVTAANIKSPTYKEFEVLSGNQVSVRFGVNNATTSPGTNLYIVGNVNELGNWDADKAIGPMFNQVMYQYPTWYYDISVPAGKNLEYKYIKKDQNGNVVWQSGNNRTYTSPTTGTDTVMINW

SEQ ID NO: 15 - protein, 711 aa, Bacillus stearothermophilus
MRRWLSLVLSMSFVFSAIFIVSDTQKVTVEAAGNLNKVNFTSDVVYQIVVDRFVDGNTSNNPSGALFSSGCTNLRKYCGGDWQGIINKINDGYLTDMGVTAIWISQPVENVFSVMNDASGSASYHGYWARDFKKPNPFFGTLSDFQRLVDAAHAKGIKVIIDFAPNHTSPASETNPSYMENGRLYDNGTLLGGYTNDANMYFHHNGGTTFSSLEDGIYRNLFDLADLNHQNPVIDRYLKDAVKMWIDMGIDGIRMDAVKHMPFGWQKSLMDEIDNYRPVFTFGEWFLSENEVDANNHYFANESGMSLLDFRFGQKLRQVLRNNSDNWYGFNQMIQDTASAYDEVLDQVTFIDNHDMDRFMIDGGDPRKVDMALAVLLTSRGVPNIYYGTEQYMTGNGDPNNRKMMSSFNKNTRAYQVIQKLSSLRRNNPALAYGDTEQRWINGDVYVYERQFGKDVVLVAVNRSSSSNYSITGLFTALPAGTYTDQLGGLLDGNTIQVGSNGSVNAFDLGPGEVGVWAYSATESTPIIGHVGPMMGQVGHQVTIDGEGFGTNTGTVKFGTTAANVVSWSNNQIVVAVPNVSPGKYNITVQSSSGQTSAAYDNFEVLTNDQVSVRFVVNNATTNLGQNIYIVGNVYELGNWDTSKAIGPMFNQVVYSYPTWYIDVSVPEGKTIEFKFIKKDSQGNVTWESGSNHVYTTPTNTTGKIIVDWQN

SEQ ID NO: 16 - protein, 655 aa, Klebsiella pneumoniae
MKRNRFFNTSAAIAISIALNTFFCSMQTIAAEPEETYLDFRKETIYFLFLDRFSDGDPSNNAGFNSATYDPNNLKKYTGGDLRGLINKLPYLKSLGVTSIWITPPIDNVNNTDAAGNTGYHGYWGRDYFRIDEHFGNLDDFKELTSLMHSPDYNMKLVLDYAPNHSNANDENEFGALYRDGVFITDYPTNVAANTGWYHHNGGVTNWNDFFQVKNHNLFNLSDLNQSNTDVYQYLLDGSKFWIDAGVDAIRIDAIKHMDKSFIQKWTSDIYDYSKSIGREGFFFFGEWFGASANTTTGVDGNAIDYANTSGSALLDFGFRDTLERVLVGRSGNTMKTLNSYLIKRQTVFTSDDWQVVFMDNHDMARIGTALRSNATTFGPGNNETGGSQSEAFAQKRIDLGLVATMTVRGIPAIYYGTEHYAANFTSNSFGQVGSDPYNREKMPGFDTESEAFSIIKTLGDLRKSSPAIQNGTYTELWVNDDILVFERRSGNDIVIVALNRGEANTINVKNIAVPNGVYPSLIGNNSVSVANKRTTLTLMQNEAVVIRSQSDDAENPTVQSINFTCNNGYTISGQSVYIIGNIPQLGGWDLTKAVKISPTQYPQWSASLELPSDLNVEWKCVKRNETNPTANVEWQSGANNQFNSNDTQTTNGSF

SEQ ID NO: 17 - protein, 686 aa, Bacillus stearothermophilus
SSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 18 - protein, 7 aa, Artificial sequence
  Synthetic construct
TLAGTDN

SEQ ID NO: 19 - protein, 11 aa, Artificial sequence
  Synthetic construct
YGDDPGTANHL

SEQ ID NO: 20 - protein, 12 aa, Artificial sequence
  Synthetic construct
YGDDPGTANHLE

(19) United States
(12) Patent Application Publication
(10) Pub. No.: US 2010/0183766 A1
(43) Pub. Date: Jul. 22, 2010

(54) CGTASE VARIANTS

(75) Inventors: Allan Svendsen, Hoersholm (DK); Lars Beier, Lyngby (DK); Tina Spendler, Malov (DK); Morten Tovborg Jensen, Vaerlose (DK); Christel Thea Jorgensen, Lyngby (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/729,877

(22) Filed: Mar. 23, 2010

Related U.S. Application Data
(63) Continuation of application No. 10/562,021, filed on Jan. 11, 2006.
(60) Provisional application No. 60/484,004, filed on Jul. 1, 2003.

(30) Foreign Application Priority Data
Jul. 1, 2003 (DK) PA 2003 00994

(51) Int. Cl. A21D 2/00; C12N 9/10; C07H 21/04

(52) U.S. Cl. 426/18; 435/193; 536/23.2

(57) ABSTRACT

The inventors have developed a method of modifying the amino acid sequence of a CGTase to obtain variants. The variants may form linear oligosaccharides as an initial product by starch hydrolysis and a reduced amount of cyclodextrin and may be useful for anti-staling in baked products. The method is based on a comparison of three-dimensional (3D) structures of the CGTase with the structure of a maltogenic alpha-amylase where one or both models includes a substrate. The invention also provides novel CGTase variants.

### CROSS-REFERENCE TO RELATED APPLICATIONS

This application is continuation of U.S. application Ser. no. 10/562,021 filed on Jan. 11, 2006, which is a 35 U.S.C. 371 national application of PCT/DK2004/000468 filed Jul. 1, 2004 which claims priority or the benefit under 35 U.S.C. 119 of Danish application no. PA 2003 00994 filed Jul. 1, 2003 and U.S. provisional application No. 60/484,004 filed Jul. 1, 2003, the contents of which are fully incorporated herein by reference.

### FIELD OF THE INVENTION

The present invention relates to the construction of variants of cyclodextrin glucanotransferases (CGTases), in particular variants having the ability to form linear oligosaccharides.

### BACKGROUND OF THE INVENTION

Pdb files 1CDG, 1PAM, 1CYG and 1CIU (available at rcsb.org) show the amino acid sequences and three-dimensional structures of several cyclodextrin glucanotransferases (CGTases). WO 9943794 shows the amino acid sequence and three-dimensional structure of a maltogenic alpha-amylase from Bacillus stearothermophilus, known as Novamyl®.

Variants of a cyclodextrin glucanotransferase (CGTase) have been described in the prior art: WO 2004026043. WO 9943793. R. J. Leemhuis: “What makes cyclodextrin glycosyltransferase a transglycosylase”, University Library Groningen, 2003. H. Leemhuis et al., Journal of Biotechnology, 103 (2003), 203-212. H. Leemhuis et al., Biochemistry, 2003, 42, 7518-7526.

L. Beier et al., Protein Engineering, vol 13, no. 7, pp. 509-513, 2000 is titled “Conversion of the maltogenic α-amylase Novamyl into a CGTase”.

### SUMMARY OF THE INVENTION

The inventors have developed a method of modifying the amino acid sequence of a CGTase to obtain variants. The variants may form linear oligosaccharides as an initial product by starch hydrolysis and a reduced amount of cyclodextrin and may be useful for anti-staling in baked products. The method is based on a comparison of three-dimensional (3D) structures of the CGTase with the structure of a maltogenic alpha-amylase where one or both models includes a substrate. The invention also provides novel CGTase variants.

Accordingly, the invention provides a method of producing a variant polypeptide, which method comprises:

a) providing an amino acid sequence and a three-dimensional model for a cyclodextrin glucanotransferase (CGTase) and for an amino acid sequence for a maltogenic alpha-amylase wherein one or both models includes a substrate,

b) superimposing the two three-dimensional models,

c) selecting an amino acid residue in the CGTase which:
i) has a C-alpha atom located >0.8 Å from the C-alpha atom of any amino acid residue in the maltogenic alpha-amylase and is located <10 Å from an atom of a substrate,
ii) has a C-alpha atom located <6 Å from a non-H atom of an amino acid residue of the maltogenic alpha-amylase corresponding to residue 190-194 of SEQ ID NO: 17, or
iii) is in a subsequence (a “loop”) of the CGTase wherein each residue has a C-alpha atom located >0.8 Å from the C-alpha atom of any residue in the maltogenic alpha-amylase sequence and wherein at least one CGTase residue has a C-alpha atom located <10 Å from a substrate, or is among the three amino acids adjacent to such subsequence in the amino acid sequence,

d) modifying the CGTase sequence wherein the modification comprises substitution or deletion of the selected residue or by insertion of a residue adjacent to the selected residue, and

e) producing the polypeptide having the resulting amino acid sequence.

The invention also provides a variant polypeptide which has an amino acid sequence with at least 70% identity to SEQ ID NO: 6; and has the ability to form linear oligosaccharides as an initial product when acting on starch.

Compared to SEQ ID NO: 6, the variant polypeptide may comprise at least one additional amino acid in a region corresponding to amino acids 194-198 and have a different amino acid or an insertion or deletion at a position corresponding to amino acid 16, 47, 85-95, 117, 139, 145, 146, 152, 153, 168, 169, 174, 184, 191, 260-269, 285, 288, 298, 314, 335, 413, 556, 602 or 677.

Alternatively, compared to SEQ ID NO: 6 the variant polypeptide may comprise at least one additional amino acid in a region corresponding to amino acids 260-269 and have a different amino acid or an insertion or deletion at a position corresponding to amino acid 16, 47, 85-95, 117, 139, 145, 146, 152, 153, 168, 169, 174, 181, 184, 191, 194, 285, 288, 298, 314, 335, 413, 556, 602 or 677.

### BRIEF DESCRIPTION OF DRAWINGS

FIG. 1 shows an alignment of various known CGTase sequences. Details are given below.

FIG. 2 shows the results of a comparison of the 3D structures 1a47 for a CGTase (SEQ ID NO: 5) and 1qho for the maltogenic alpha-amylase Novamyl (SEQ ID NO: 17). Details are described in Example 1.

### DETAILED DESCRIPTION OF THE INVENTION

### CGTase

The method of the invention uses an amino acid sequence of a CGTase and a three-dimensional model for the CGTase. The CGTase may have a catalytic triad, and the model may include a substrate.

The CGTase may have a three-dimensional structure found under the indicated identifier in the Protein Data Bank (www.rcsb.org): B. circulans (1CDG), alkalophilic Bacillus (1PAM), B. stearothermophilus (1CYG) or Thermoanaerobacterium thermosulfurigenes (1CIU, 1A47). 3D structures for other CGTases may be constructed as described in Example 1 of WO 9623874.

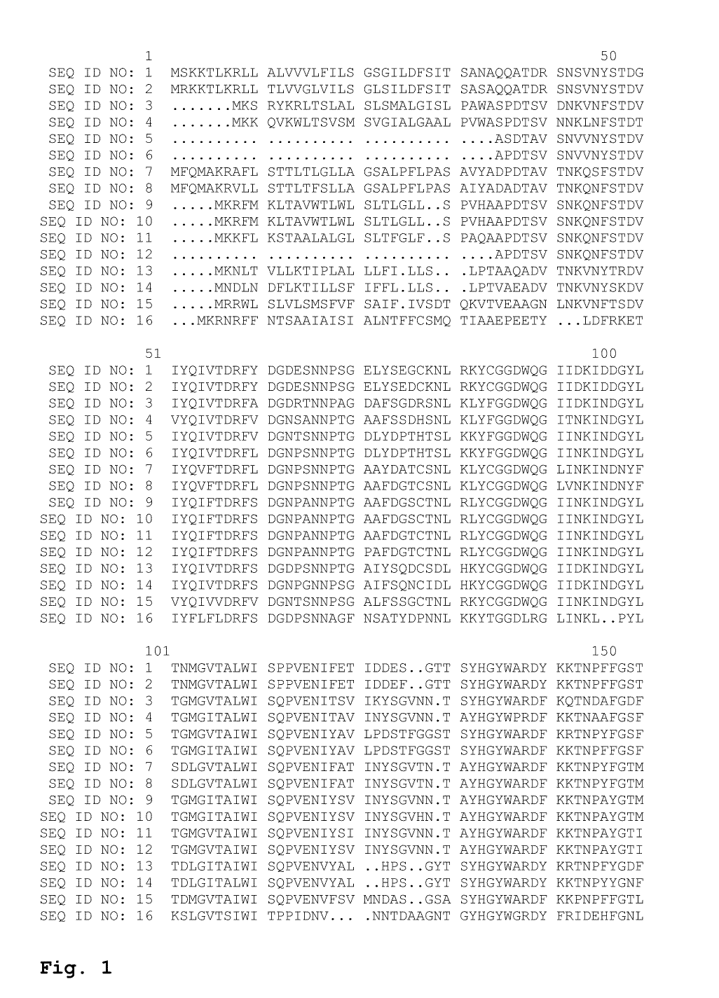

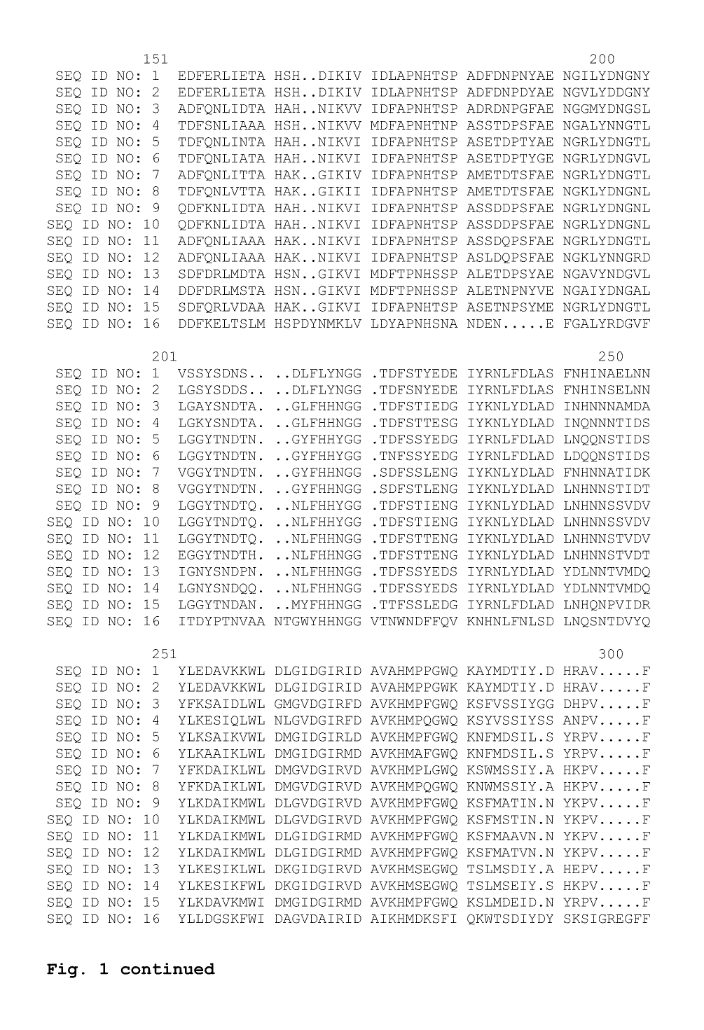

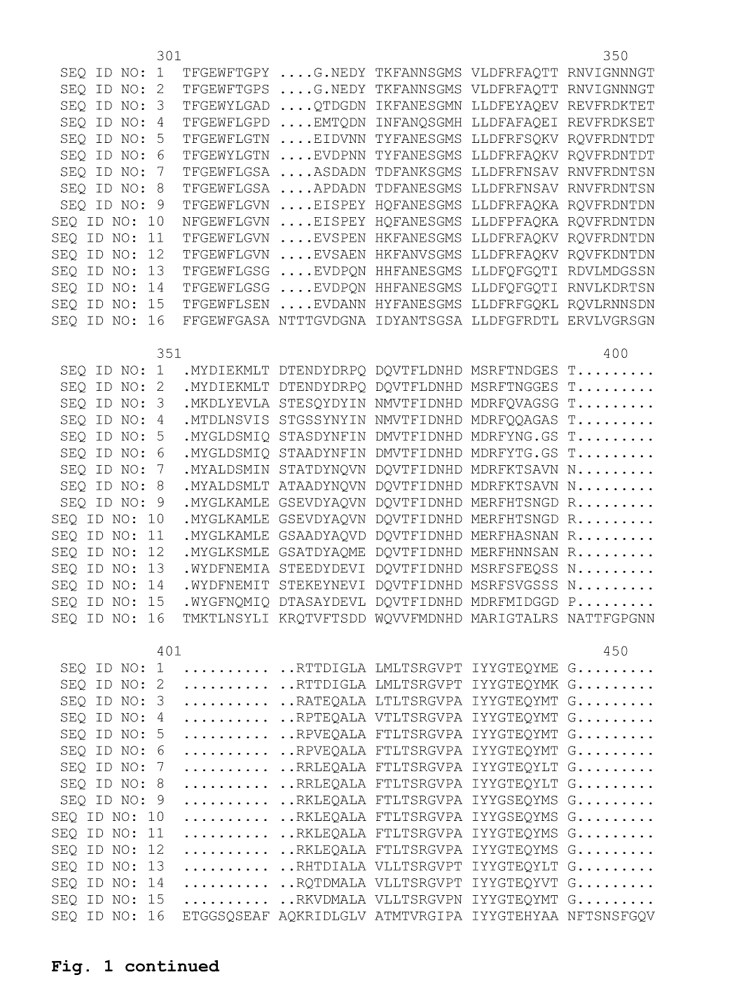

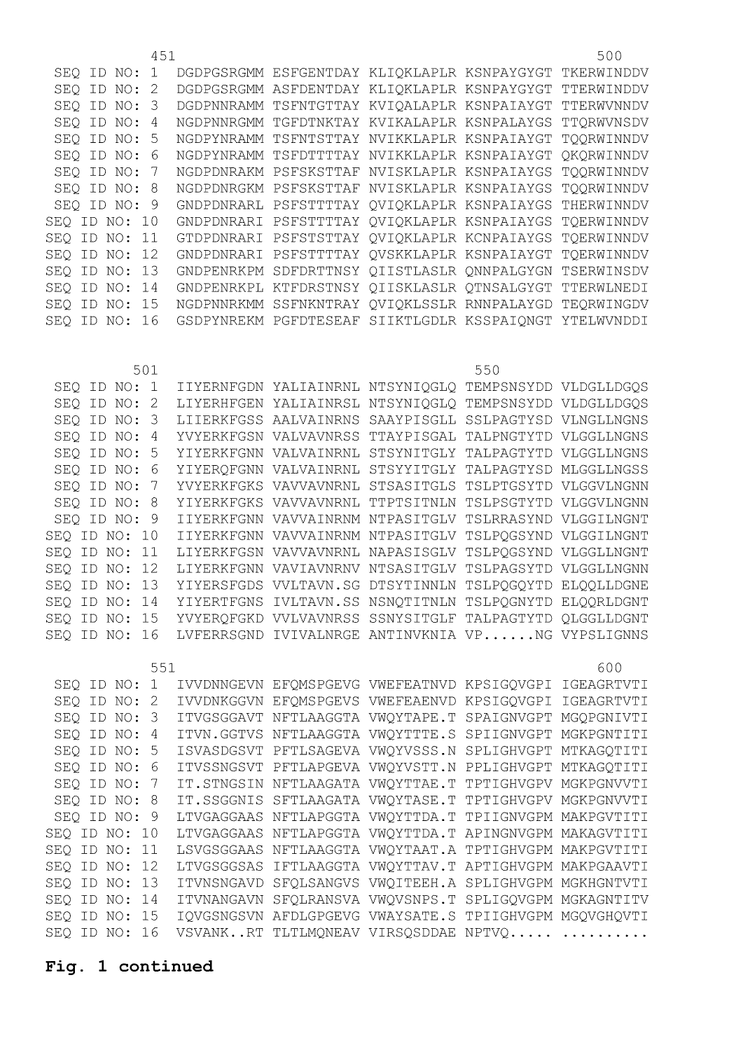

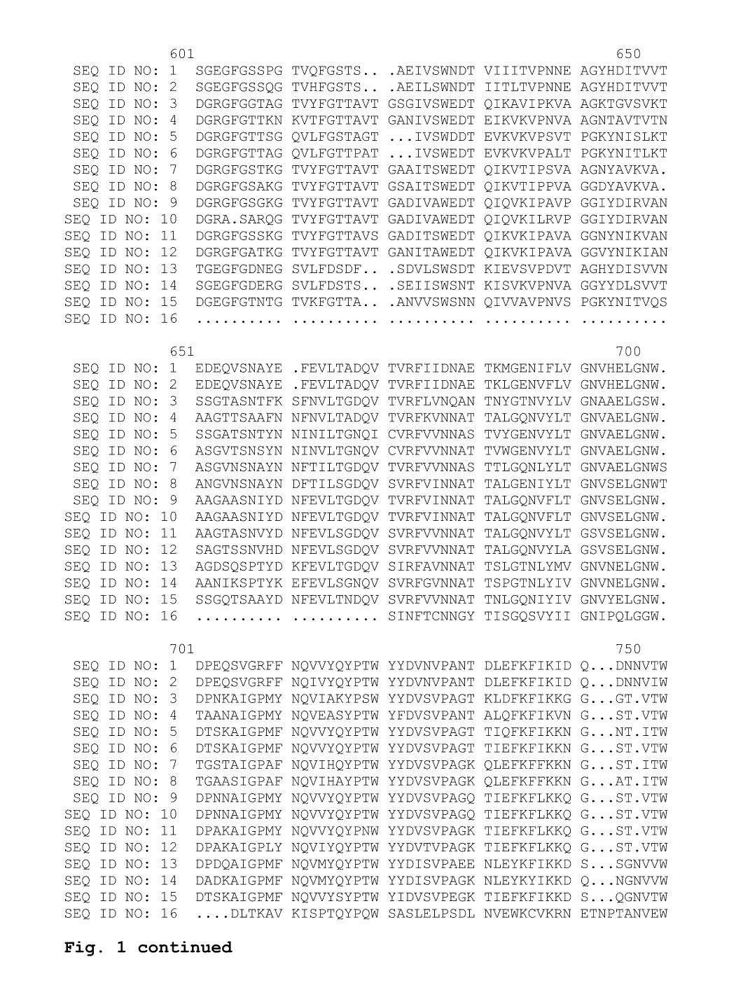

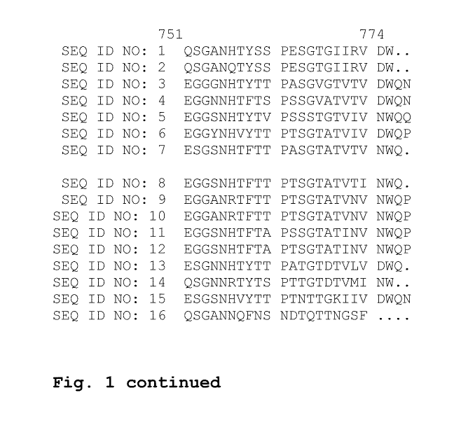

FIG. 1 shows an alignment of the following known CGTase sequences, each identified by accession number in the GeneSeqP database and by source organism. Some sequences include a propeptide, but only the mature peptide is relevant for this invention.

SEQ ID NO: 1. aab71493.gcg B. agaradherens

SEQ ID NO: 2. aau76326.gcg Bacillus agaradhaerans

SEQ ID NO: 3. cdg1_paema.gcg Paenibacillus macerans (Bacillus macerans).

SEQ ID NO: 4. cdg2_paema.gcg Paenibacillus macerans (Bacillus macerans).

SEQ ID NO: 5. cdgt_thetu.gcg Thermoanaerobacter thermosulfurogenes (Clostridium thermosulfurogenes) (SEQ ID NO: 2:)

SEQ ID NO: 6. aaw06772.gcg Thermoanaerobacter thermosulphurigenes sp. ATCC 53627 (SEQ ID NO: 3)

SEQ ID NO: 7. cdgt_bacci.gcg Bacillus circulans

SEQ ID NO: 8. cdgt_bacli.gcg Bacillus sp. (strain 38-2)

SEQ ID NO: 9. cdgt_bacs0.gcg Bacillus sp. (strain 1011)

SEQ ID NO: 10. cdgt_bacs3.gcg Bacillus sp. (strain 38-2)

SEQ ID NO: 11 cdgu_bacci.gcg Bacillus circulans

SEQ ID NO: 12. cdgt_bacsp.gcg Bacillus sp. (strain 17-1, WO 2003068976) (SEQ ID NO: 4)

SEQ ID NO: 13. cdgt_bacoh.gcg Bacillus ohbensis

SEQ ID NO: 14. cdgt_bacs2.gcg Bacillus sp. (strain 1-1)

SEQ ID NO: 15. cdgt_bacst.gcg Bacillus stearothermophilus

SEQ ID NO: 16. cdgt_klepn.gcg Klebsiella pneumoniae

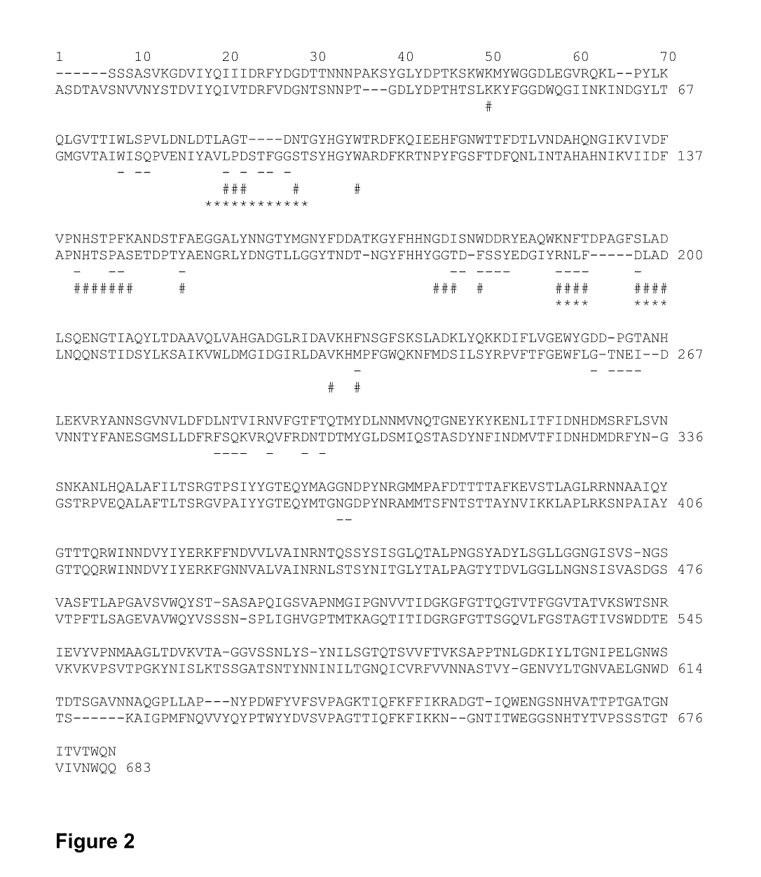

To develop variants of a CGTase without a known 3D structure, the sequence may be aligned with a CGTase having a known 3D structure. An alignment for a number of CGTase sequences is shown in FIG. 2. Other sequences may be aligned by conventional methods, e.g. by use the software GAP from UWGCG Version 8.

### Maltogenic Alpha-Amylase

The method also uses an amino acid sequence of a maltogenic alpha-amylase (EC 3.2.1.133) and a three-dimensional model of the maltogenic alpha-amylase. The maltogenic alpha-amylase may have a catalytic triad, and the model may include a substrate. The maltogenic alpha-amylase may have the amino acid sequence shown in SEQ ID NO: 17 (in the following referred to as Novamyl). A 3D model for Novamyl with a substrate is described in U.S. Pat. No. 6,162,628 and is found in the Protein Data Bank with the identifier 1QHO. Alternatively, the maltogenic alpha-amylase may be a Novamyl variant described in U.S. Pat. No. 6,162,628. A 3D structure of such a variant may be developed from the Novamyl structure by known methods, e.g. as described in T. L. Blundell et al., Nature, vol. 326, p. 347 ff (26 Mar. 1987); J. Greer, Proteins: Structure, Function and Genetics, 7:317-334 (1990); or Example 1 of WO 9623874.

### Superimposition of 3D Models

The two 3D models may be superimposed by aligning the amino acid residues of each catalytic triad. This may be done by methods known in the art based on the deviations of heavy atoms in the two triads, e.g. by minimizing the sum of squares of deviations. Alternatively, the superimposition may be done so as to keep deviations between corresponding atoms below 0.8 Å, e.g. below 0.6 Å, below 0.4 Å, below 0.3 Å or below 0.2 Å.

Alternatively, the superimposition may be based on the deviations of all corresponding pairs of amino acid residues as shown in the alignment in FIGS. 4-5 of WO 9943793 and bringing the sum of square of all deviations to a minimum.

### Selection of Amino Acid Sequences

In the superimposed 3D models, amino acid residues in the CGTase sequence are selected if they meet at least one of three conditions:

The CGTase residue has a C-alpha atom located >0.8 Å from the C-alpha atom of any amino acid residue in the maltogenic alpha-amylase, and it is located <10 Å from an atom of a substrate.

The CGTase residue has a C-alpha atom located <6 Å from a heavy atom (i.e., an atom other than H) of an amino acid residue of the maltogenic alpha-amylase corresponding to residue 190-194 of SEQ ID NO: 17.

The CGTase residue is in a subsequence (a “loop”) of the CGTase or in the “pre-fix” or “post-fix” of the loop. The CGTase loop is a subsequence wherein each residue has a C-alpha atom located >0.8 Å from the C-alpha atom of any residue in the maltogenic alpha-amylase sequence, and at least one CGTase residue of the loop has a C-alpha atom located <10 Å from a substrate. The pre-fix and post-fix are defined as three amino acid residues in the sequence before and after the loop.

The selected CGTase residue may correspond to residue 47, 75, 77, 78, 85, 86, 87, 88, 89, 90, 91, 92, 93, 94, 95, 96, 102, 139, 140, 141, 142, 143, 144, 145, 146, 152, 153, 168, 169, 180, 181, 182, 183, 184, 185, 186, 187, 191, 193, 194, 195, 196, 197, 198, 199, 200, 231, 234, 235, 262, 263, 264, 265, 266, 286, 287, 288, 289, 292, 296, 298, 335, 353, 369, 370, 413, or 556 of SEQ ID NO: 5.

### Modifications of CGTase Amino Acid Sequence

A selected CGTase residue may be deleted or may be substituted with a different residue. The substitution may be made with the same amino acid residue as found at a corresponding position in an alignment with the maltogenic alpha-amylase sequence or with a residue of the same type. The type indicates a positively charged, negatively charged, hydrophilic or hydrophobic residue, understood as follows (Tyr may be hydrophilic or hydrophobic):

Hydrophobic amino acids: Ala, Val, Leu, Ile, Pro, Phe, Trp, Gly, Met, Tyr

Hydrophilic amino acids: Thr, Ser, Gln, Asn, Tyr, Cys

Positively charged amino acids: Lys, Arg, His

Negatively charged amino acids: Glu, Asp

The substitution of the CGTase residue may be with a larger or smaller residue depending on whether a larger or smaller residue is found at a corresponding position in the maltogenic alpha-amylase sequence. In this connection, the residues are ranked as follows from smallest to largest: (an equal sign indicates residues with sizes that are practically indistinguishable):

G<A=S=C<V=T<P<L=I=N=D=M<E=Q<K<H<R<F<Y<W

One or more amino acid residues may be inserted at a position adjacent to the selected CGTase residue on the amino or carboxyl side. The insertion may be made at a position in the CGTase sequence where the maltogenic amylase contains additional residues, and the insertion may consist of an equal number of residues, or the insertion may have one or two fewer or more residues. Each inserted residue may be the same as the corresponding maltogenic amylase residue or of the same type.

The insertion may particularly be made at a position corresponding to residues in the regions 85-96, 193-200 or 260-269 of SEQ ID NO: 5. The insertion at residues 193-200 may particularly consist of 1-7 residues, e.g. 1, 2, 3, 4, 5, 6 or 7 residues, and may particularly consist of DPAGF, e.g. between residues 196 and 197 of SEQ ID NO: 5, and it may be combined with a substitution corresponding to L195F, F196T and D197S in SEQ ID NO: 5.

More particularly, the modification may comprise substitution of amino acids corresponding to amino acids 85-95, 260-268 or 260-269 of SEQ ID NO: 5 or 6 with TLAGTDN (SEQ ID NO:18), YGDDPGTANHL (SEQ ID NO:19) or YGDDPGTANHLE (SEQ ID NO: 20), respectively.

The substitution may correspond to V16A, K47K, T117R, P139L, A145F, F146K, Y152F, G153V/G, Y168F, T169I, G174S, G181D, F184W, I191T, N194S, R285D, Q288T, T298, D314E, T335A, R353H, W413R, G556S, Y602L, or V677K of SEQ ID NO: 5 or 6.

### Optional Further Modifications of the CGTase Sequence

Optionally, the CGTase sequence may be further modified by substituting one or more residues which is not selected. The substitution may be made with an amino acid residue of the same type (in particular with the same residue) as the corresponding residue in an alignment with the maltogenic alpha-amylase sequence.

Depending on whether the matching residue in the maltogenic alpha-amylase sequence is smaller or larger than the residue in the CGTase sequence, the substitution may be made with a smaller or larger residue (using the ranking shown above).

### Production of CGTase Variants

A polypeptide having the resulting amino acid sequence may be produced by conventional methods, generally involving producing DNA with a sequence encoding the polypeptide together with control sequences, transforming a suitable host organism with the DNA, cultivating the transformed organism at suitable conditions for expressing and optionally secreting the polypeptide, and optionally recovering the expressed polypeptide, e.g. as described in WO 9943793.

DNA encoding any of the above CGTase variants may be prepared, e.g. by point-specific mutation of DNA encoding the parent CGTase. This may be followed by transformation of a suitable host organism with the DNA, and cultivation of the transformed host organism under suitable conditions to express the encoded polypeptide (CGTase variant). This may be done by known methods.

### Properties of CGTase Variants

The CGTase variants of the invention may form linear oligosaccharides as an initial product by starch hydrolysis and a reduced amount of cyclodextrin and may be useful for anti-staling in baked products. The modification of the amino acid sequence according to the invention may result in reduced cyclization and disproportionation activities and an increased ratio of hydrolysis/cyclization activities, measured, e.g., as described by H. Leemhuis, Journal of Biotechnology, 103 (2003), 203-212.

Optionally, one or more expressed polypeptides may be tested for one or more useful enzymatic activities, and a variant may be selected accordingly. Thus, the ability to hydrolyze starch or a starch derivative may be tested by a conventional method, e.g. a plate assay, use of Phadebas tablets or DSC on amylopectin. Further, the initial product from starch hydrolysis may be analyzed and a polypeptide producing an increased ratio of linear oligosaccharides to cyclodextrins may be selected. The initial product may have a high ratio of maltose or maltose+glucose (G2 or G1+G2) compared to total dextrins (maltooligosaccharides G1−G7 or G1−G7+cyclodextrins). This may be measured as described in an example.

Also, the polypeptide may be tested by adding it to a dough, baking it and testing the firmness of the baked product during storage; a polypeptide with anti-staling effect may be selected as described in WO 9104669 or U.S. Pat. No. 6,162,628.

The substitutions according to the invention may improve the thermostability of the

CGTase variants. Variants may be screened for their thermostability, e.g. by DSC (differential scanning calorimetry) at pH 5.5 in 0.1 M Na acetate, scan rate 90 K/h, and a variant with an improved thermostability may be selected. The substitutions may also increase the yield when expressed in a suitable transformed host organism; this may be edxplained by an improved stability.

Optionally, the amino acid sequence may be further modified to improve the properties of the variant, particularly to improve its thermostability. Such modification may include amino acid substitutions similar to those described in U.S. Pat. No. 6,162,628 or in H. Leemhuis et al., Proteins: Structure, Function and Bioinformatics, 54:128-134 (2004).

### Optional Gene Recombination

Optionally, DNA encoding a plurality of the above CGTase variants may be prepared and recombined, followed by transformation of a suitable host organism with the recombined DNA, and cultivation of the transformed host organism under suitable conditions to express the encoded polypeptides (CGTase variants). The gene recombination may be done by known methods.

### CGTase Variants

Particularly, the CGTase may be modified by substitution, insertion or deletion of an amino acid at a position corresponding to amino acid 85-95, 152, 184, 260-269, 285, 288, 314 of the amino acid sequence shown in SEQ ID NO: 5 or 6. The modification may comprise substitution or insertion of an amino acid residue with an amino acid residue of a corresponding position in the amino acid sequence of Novamyl (SEQ ID NO: 17) or a deletion of an amino acid residue in the region which is not present at the corresponding position in the Novamyl sequence.

More particularly, the modification may comprise substitution of amino acids corresponding to amino acids 85-95, 260-268 or 260-269 of SEQ ID NO: 5 or 6 with TLAGTDN (SEQ ID NO:18), YGDDPGTANHL (SEQ ID NO: 19) or YGDDPGTANHLE (SEQ ID NO:20), respectively.

Some particular examples with the Thermoanaerobacter CGTase (SEQ ID NO: 6) as an example are Y152F, F184W, R285D, Q288T, D314E. Corresponding substitutions may be made in other CGTases.

Also, one or more additional modifications may be made, each being an amino acid substitution, insertion or deletion. In particular, such modification may be made in the regions corresponding to amino acids 40-43, 78-85, 136-139, 173-180, 189-195 or 258-268 of SEQ ID NO: 17. In particular, the modification may be an insertion of or a substitution with an amino acid present at the corresponding position of Novamyl, or a deletion of an amino acid not present at the corresponding position of Novamyl. Thus, taking the Thermoanaerobacter CGTase (SEQ ID NO: 6) as an example, one or more of the following changes may be made to introduce a loop modeled on Novamyl:

A85-S95 of SEQ ID NO: 6 is replaced by T80-N86 of SEQ ID NO: 17,

N194-L198 of SEQ ID NO: 6 is replaced by N187-L196 of SEQ ID NO: 17,

Y260-P268 of SEQ ID NO: 6 is replaced by Y258-L268 of SEQ ID NO: 17, or

Y260-N269 of SEQ ID NO: 6 is replaced by Y258-E269 of SEQ ID NO: 17.

### Examples

### Example 1

### Construction of CGTase Residues Based on 3D Structures

Two 3D structures with substrates were used: 1A47 for a CGTase (SEQ ID NO: 5) and 1 QHO for a maltogenic alpha-amylase (Novamyl, SEQ ID NO: 17), wherein the substrates are indicated as GTE, GLC, CYL and GLD for 1a47 and as ABD for 1 qho. The two structures were superimposed by minimizing the sum of squares for deviations at the three C-alpha atoms at the catalytic triad: D230, E258 and D329 for 1A47, and D228, E256 and D329 for Novamyl. The superimposed structures were analyzed, and the result is shown in FIG. 2 with the Novamyl sequence at the top and the CGTase sequence below.

The following CGTase residues were found to have a C-alpha atom <10 Å from an atom of either substrate: 19, 21, 24, 46-47, 75, 77-78, 82-83, 85-103, 106, 136-145, 152-153, 182-187, 190-191, 193-200, 228-235, 257-267, 270, 282-289, 291-292, 296, 298, 324, 327-331, 359, 369-375. Out of these, the following were found to have a C-alpha atom >0.8 Å from the C-alpha atom of any Novamyl residue: 75, 77-78, 87, 89, 91-92, 94, 140, 144-145, 152, 182-187, 193-197, 235, 262-266, 286-289, 292, 296, 298, 369-370. They are indicated by underlining in FIG. 2.

The following CGTase residues were found to have a C-alpha atom <6 Å from an atom other than hydrogen (a “heavy” atom) of one of the Novamyl residues 190-194: 47, 87-89, 95, 102, 140-146, 152, 180-182, 184, 193-200, 231, 234. They are marked by # in FIG. 2.

Two subsequences (“loops”) of consecutive CGTase residues were identified where some residues have the C-alpha atom <10 Å from an atom of either substrate and >0.8 Å from the C-alpha atom of any Novamyl residue. Including prefix and postfix (3 residues each), the two subsequences are at residues 85-96 and 193-200 of the CGTase. They are indicated by asterisks in FIG. 2.

To construct variants of the CGTase of SEQ ID NO: 6, the corresponding residues were identified in the alignment in FIG. 1. As a result of the high degree of identity, the residues have the same numbers in the two sequences. Variants were constructed, each having one or more loops modeled on Novamyl together with one or more substitutions, as follows:

Novamyl T80-N86: 85A*, 86V*, 87L*, 88P*, D89T, S90L, T91A, F92G, G93T, G94D

Novamyl G259-L268: *260aG, *260bD, L261D, G262P, T263G, N264T, E265A, V266N, D267H, P268L

Novamyl F188-5195: *194aF, *194bT, *194cD, *194dP, *194eA, L195G, D197S

| Novamyl loops | Additional substitutions |
|---|---|
| T80-N86, F188-S195 | Y152F |
| T80-N86, F188-S195, G259-L268 | Y152F, D314E |
| T80-N86, F188-S195, G259-L268 | Y152F, F184W, R285D, Q288T, D314E |
| T80-N86, G259-L268 | Y152F, G257D, R285D, Q288T, D314E |
| T80-N86, G259-L268 | Y152F, R285D, Q288T, D314E |
| T80-N86, G259-L268 | A145F, Y152F, R285D, Q288T, D314E |
| T80-N86, G259-L268 | S146K, Y152F, R285D, Q288T, D314E |
| T80-N86, G259-L268 | A145F, S146K, Y152F, G257D, R285D, Q288T, D314E |
| T80-N86, F188-S195, G259-L268 | A145F, Y152F, F184W, R285D, Q288T, D314E |
| T80-N86, F188-S195, G259-L268 | S146K, Y152F, F184W, R285D, Q288T, D314E |
| T80-N86, F188-S195, G259-L268 | A145F, S146K, Y152F, F184W, R285D, Q288T, D314E |
| T80-N86 | Y152F, T207N |
| T80-N86, G259-L268 | A145F, Y152F, R285D, Q288T, D314E |
| T80-N86, G259-L268 | S146K, Y152F, R285D, Q288T, D314E |
| T80-N86, G259-L268 | A145F, S146K, Y152F, R285D, Q288T, D314E |
| T80-N86, G259-L268 | Y152F, F196G, G257D, R285D, Q288T, D314E |
| T80-N86, G259-L268 | Y152F, F196G, R285D, Q288T, D314E |
| T80-N86, G259-L268 | Y152F, F184N, F196G, G257D, R285D, Q288T, D314E |
| T80-N86, G259-L268 | Y152F, F184N, F196G, R285D, Q288T, D314E |
| T80-N86, F188-S195, G259-L268 | Y152F, R285D, Q288T, D314E |
| T80-N86, F188-S195, G259-L268 | A145F, Y152F, R285D, Q288T, D314E |
| T80-N86, F188-S195, G259-L268 | S146K, Y152F, R285D, Q288T, D314E |
| T80-N86, F188-S195, G259-L268 | A145F, S146K, Y152F, R285D, Q288T, D314E |
| T80-N86, F188-S195, G259-L268 | Y152F, G181D, F184W, R285D, Q288T, D314E |
| T80-N86, G259-L268 | Y152F, G181D, F184W, G257D, R285D, Q288T, D314E |
| T80-N86, G259-L268 | A145F, Y152F, G181D, F184W, R285D, Q288T, D314E |
| T80-N86, G259-L268 | S146K, Y152F, G181D, F184W, R285D, Q288T, D314E |
| T80-N86, G259-L268 | A145F, S146K, Y152F, G181D, F184W, G257D, |
| | R285D, Q288T, D314E |
| T80-N86, F188-S195, G259-L268 | A145F, Y152F, G181D, F184W, R285D, Q288T, D314E |
| T80-N86, F188-S195, G259-L268 | S146K, Y152F, G181D, F184W, R285D, Q288T, D314E |
| T80-N86, F188-S195, G259-L268 | A145F, S146K, Y152F, G181D, F184W, R285D, Q288T, |
| | D314E |
| T80-N86, F188-S195, G259-L268 | Y152F, G181D, R285D, Q288T, D314E |
| T80-N86, F188-S195, G259-L268 | A145F, Y152F, G181D, R285D, Q288T, D314E |
| T80-N86, F188-S195, G259-L268 | S146K, Y152F, G181D, R285D, Q288T, D314E |
| T80-N86, F188-S195, G259-L268 | A145F, S146K, Y152F, G181D, R285D, Q288T, D314E |
| T80-N86, G259-L268 | Y152F, G181D G257D, R285D, Q288T, D314E |
| T80-N86, G259-L268 | A145F, Y152F, G181D, R285D, Q288T, D314E |
| T80-N86, G259-L268 | S146K, Y152F, G181D, R285D, Q288T, D314E |
| T80-N86, G259-L268 | A145F, S146K, Y152F, G181D, G257D, R285D, Q288T, |
| | D314E |
| T80-N86, F188-S195, G259-L268 | A145F, S146K, Y152F, G181D, F184W, R285D, Q288T, |
| | D314E, F384S |

Similarly, variants of the CGTase of SEQ ID NO: 12 were constructed, each having modifications to emulate the following three Novamyl loops:

T80-D85: 85S*,86V*, 87I*, N88T, Y89L, S90A, V92T, N93D

F188-S195: L194F, Y195T, *196aP, *196bA, *196cG, *196dF, *196eS

Y258-L268: “258aY, *258bG, F259D, L260D, G261P, V262G, N263T, E264A, I265N, S266H, P267L”

| Novamyl loops | Additional substitutions |
|---|---|
| T80-D85, F188-S195, Y258-L268 | N173S |
| T80-D85, F188-S195, Y258-L268 | R284D, Q287T, D313E, F605L |
| T80-D85, F188-S195, Y258-L268 | Q116R, D639G |
| T80-D85, F188-S195, Y258-L268 | V16A, Q116R, A144F, S145K, R284D, Q287T, M680K |
| T80-D85, F188-S195, Y258-L268 | A144F, S145K, R284D, Q287T, D313E |
| T80-D85, F188-S195, Y258-L268 | A144F, S145K, G180D, R284D, Q287T, D313E |
| T80-D85, F188-S195, Y258-L268 | A144F, S145K, G180D, F183W, R284D, Q287T, D313E |
| T80-D85, F188-S195, Y258-L268 | A144F, S145K, F183W, R284D, Q287T, D313E |
| T80-D85, F188-S195, Y258-L268 | R47K, A144F, S145K, R284D, Q287T, D313E |
| T80-D85, F188-S195, Y258-L268 | R47K, A144F, S145K, G1800, R284D, Q287T, D313E |
| T80-D85, F188-S195, Y258-L268 | R47K, A144F, S145K, G180D, F183W, R284D, Q287T, |
| | D313E |
| T80-D85, F188-S195, Y258-L268 | R47K, A144F, S145K, F183W, R284D, Q287T, D313E |
| T80-D85, F188-S195, Y258-L268 | Q116R, P138L, A144F, S145K, A152V, I190T, T334A, |
| | R353H |
| T80-D85, F188-S195, Y258-L268 | A144F, S145K, Y167F, T168I, N173S, N193S, T297I, |
| | G559S |
| T80-D85, F188-S195, Y258-L268 | A144F, S145K, A152G, W413R, F605L |

### Example 2

### Starch Hydrolysis with CGTase Variants

Nine variants prepared in Example 1 were tested to determine the initial product profile in starch hydrolysis. The variants including 7 variants of SEQ ID NO: 6 and 2 variants of SEQ ID NO: 12. The two parent CGTases were tested for comparison.

Incubations were carried out using 2% amylopectin (potato starch) in 50 mM NaOAc, pH 5.7, 5 mM CaCl2. Crude culture broth (20-100 micro-L) was added to the substrate solution (900-980 micro-L), and the mixture incubated at 40° C. or 60° C. and the conversion was followed by TLC (TLC eluent: acetonitrile/EtOAc, n-propanol/water 85:20:50:50, visualization: 1M H₂SO₄ followed by heating). At a detectable conversion (4-18 h), a sample (100 micro-L) was taken out and inactivated with 1M NaOH (10 micro-L). The sample was diluted (30 micro-L to 1000 micro-L MilliQ water) and filtered through 0.45 μm Millex®-HV filter before analysis by HPAEC/high-performance anion exchange chromatography).

The samples were analyzed on a Dionex DX-500 HPAEC-PAD system (CarboPac PA-100 column; A buffer: 150 mM NaOH; B buffer: 150 mM NaOH+0.6 M sodium acetate; Flow rate: 1 ml/min. Elution conditions: 0-3 min: 95% A+5% B; 3-19 min: linear gradient: 95% A+5% B to 50% A and 50% B; 19-21 min: linear gradient: 50% A+50% B to 100% B 21-23 min: 100% B). As reference on the Dionex system a mixture of maltooligosaccharides was used (DP2 to DP7, 100 micro-M of each) and α-, β- and γ-CD (100 micro-M of each). These were used to quantify the amounts of each oligosaccharide formed.

The results were expressed as G2/sum, (G1+G2)/sum and CD/sum where G1 is the peak area for glucose, G2 is the peak area for maltose, CD is the total of peak areas for alpha-, beta- and gamma-cyclodextrin, and sum is the total of peak areas for G1−G7 maltodextrins and mcyclodextrins. G2/sum was 0.12-0.68 for the variants compared to 0 or 0.03 for the parent CGTases. (G1+G2)/sum was 0.48-0.79 for the variants compared to 0 and 0.06 for the parent CGTases. CD/sum was 0.01-0.18 for the variants compared to 0.87 and 0.94 for the parent CGTases.

### Example 3

### Baking Tests with CGTase Variants

Ten variants prepared in Example 1 were purified and tested in baking, including 7 variants of SEQ ID NO: 6 and 3 variants of SEQ ID NO: 12. Doughs were made according to the straight-dough method with addition of the CGTase variant at a dosage in the range of 1-20 mg/kg. Controls were made without enzyme addition or with addition of one of the two parent CGTases.

The doughs were baked to make panned bread, and the bread was stored for a week. Firmness, elasticity and mobility of free water were measured for the bread loaves after 1, 4 and 7 days storage. A sensory ranking of moistness was made by a trained test panel for bread after 7 days.

Each of the variants was ranked better than a control without enzyme. The CGTases had a detrimental effect on elasticity, whereas the variants did not effect the elasticity negatively. The bread made with CGTase was gummy and unacceptable.

1-14. (canceled)

15. A polypeptide which:
a) has an amino acid sequence having at least 70% identity to SEQ ID NO: 6;
b) compared to SEQ ID NO: 6 comprises at least one additional amino acid in a region corresponding to amino acids 194-198,
c) compared to SEQ ID NO: 6 has a different amino acid or an insertion or deletion at a position corresponding to amino acid 16, 47, 85-95, 117, 139, 145, 146, 152, 153, 168, 169, 174, 184, 191, 260-269, 285, 288, 298, 314, 335, 413, 556, 602 and/or 677, and
d) has the ability to form linear oligosaccharides as an initial product when acting on starch.

16. The polypeptide of claim 15, which compared to SEQ ID NO: 6 comprises 1-7 additional amino acids in the region corresponding to amino acids 194-198.

17. The polypeptide of claim 15, which compared to SEQ ID NO: 6 comprises 5 additional amino acids in the region corresponding to amino acids 194-198.

18. The polypeptide of claim 15, which compared to SEQ ID NO: 6 comprises 1-7 additional amino acids between amino acids corresponding to 194 and 195 of SEQ ID NO: 6.

19. The polypeptide of claim 15, which compared to SEQ ID NO: 6 comprises 1-7 additional amino acids between amino acids corresponding to 196 and 197 of SEQ ID NO: 6.

20. The polypeptide of claim 15, which has a different amino acid from SEQ ID NO: 6 at a position corresponding to 194-198.

21. The polypeptide of claim 15, which comprises an amino acid residue which is present at the corresponding position of SEQ ID NO: 17 or deletion of an amino acid residue in SEQ ID NO: 6 which is not present at the corresponding position in the amino acid sequence shown in SEQ ID NO: 17.

22. The polypeptide of claim 15, where A85-595 of SEQ ID NO: 6 is replaced by T80-N86 of SEQ ID NO: 17, N194-L198 of SEQ ID NO: 6 is replaced by N187-L196 of SEQ ID NO: 17, Y260-P268 of SEQ ID NO: 6 is replaced by Y258-L268 of SEQ ID NO: 17, or Y260-P269 of SEQ ID NO: 6 is replaced by Y258-E269 of SEQ ID NO: 17.

23. The polypeptide of claim 15, which has TLAGTDN at positions corresponding to 85-95 of SEQ ID NO: 6, YGDDPGTANHL at 260-268 or YGDDPGTANHLE at 260-269.

24. The polypeptide of claim 15, which compared to SEQ ID NO: 6 has a substitution corresponding to V16A, K47K, T117R, P139L, A145F, F146K, Y152F, G153V/G, Y168F, T169I, G174S, G181D, F184W, I191T, N194S, R285D, Q288T, T2981, D314E, T335A, R353H, W413R, G556S, Y602L and/or V677K.

25. The polypeptide of claim 15, which has the substitution A145F.

26. The polypeptide of claim 15, which has the substitution S146K.

27. The polypeptide of claim 15, which has the substitution Y152F.

28. The polypeptide of claim 15, which has the substitution F184W.

29. The polypeptide of claim 15, which has the substitution L195G.

30. The polypeptide of claim 15, which has the substitution F196T.

31. The polypeptide of claim 15, which has the substitution D197S.

32. The polypeptide of claim 15, which has the substitution R285D.

33. The polypeptide of claim 15, which has the substitution Q288T.

34. The polypeptide of claim 15, which has the substitution D314E.

35. A polynucleotide encoding the polypeptide of claim 15.

36. A process for preparing a baked product which comprises adding the polypeptide of claim 15 and baking the dough to prepare the baked product.

37. A polypeptide which:
a) has an amino acid sequence having at least 70% identity to SEQ ID NO: 6;
b) compared to SEQ ID NO: 6 comprises at least one additional amino acid in a region corresponding to amino acids 260-269,
c) compared to SEQ ID NO: 6 has a different amino acid or an insertion or deletion at a position corresponding to amino acid 16, 47, 85-95, 117, 139, 145, 146, 152, 153, 168, 169, 174, 181, 184, 191, 194, 285, 288, 298, 314, 335, 413, 556, 602 or 677, and
d) has the ability to form linear oligosaccharides as an initial product when acting on starch.
